(19) United States
(12) Patent Application Publication
(10) Pub. No.: US 2022/0287264 A1
(43) Pub. Date: Sep. 15, 2022

(54) IDENTIFICATION INFORMATION READOUT APPARATUS, METHOD, PROGRAM, RECORDING MEDIUM, AND MEASURING SYSTEM

(71) Applicant: ADVANTEST Corporation, Tokyo (JP)

(72) Inventors: Makoto YAMAZAKI, Saitama (JP); Hoshito TAGUCHI, Gunma (JP); Tomio KUROSAWA, Saitama (JP)

(73) Assignee: ADVANTEST Corporation, Tokyo (JP)

(21) Appl. No.: 17/633,391

(22) PCT Filed: Jun. 10, 2020
(86) PCT No.: PCT/JP2020/022801

§ 371 Date: Feb. 7, 2022

(30) Foreign Application Priority Data
Sep. 18, 2019 (JP) 2019-169738

(51) Int. Cl. A01J 5/013; A01K 29/00; A01K 11/00

(52) U.S. Cl. CPC A01J 5/0132; A01K 29/005; A01K 11/006; A01K 11/004

(57) ABSTRACT

According to the present invention, an identification information readout apparatus includes an identification information readout section, a somatic cell concentration measuring section, and a measurement result recording section. The identification information readout section reads cow identification information out of a recording medium with the cow identification information recorded therein. The somatic cell concentration measuring section measures the somatic cell concentration of milk obtained from a cow. The measurement result recording section records the somatic cell concentration and the cow identification information in association with each other. The recording medium is attached to the cow.

Technical Field

The present invention relates to associating a cow with a milk measurement result.

Background Art

There has conventionally been known measuring milk to screen for a cow disease and the like (see Patent Literatures 1, 2, and 3, for example).

Citation List

Patent Literature

Patent Literature 1: Japanese Patent Application Publication No. 2002-543826

Patent Literature 2: Japanese Patent Application Publication No. 2006-518852

Patent Literature 3: Japanese Patent Application Publication No. 2015-508506

### SUMMARY OF THE INVENTION

Problem to be Solved by the Invention

It is here not possible to screen for a cow disease and the like without managing which milk measurement results correspond to which cows. It is however cumbersome to associate a milk measurement result with a cow.

It is hence an object of the present invention to make it easy to associate a milk measurement result with a cow.

Means for Solving the Problem

According to one aspect of the present invention, an identification information readout apparatus includes: an identification information readout section arranged to read cow identification information out of a recording medium with the cow identification information recorded therein; a somatic cell concentration measuring section arranged to measure the somatic cell concentration of milk obtained from a cow; and a measurement result recording section arranged to record the somatic cell concentration and the cow identification information in association with each other, wherein the recording medium is attached to the cow.

According to the thus constructed identification information readout apparatus, an identification information readout section reads cow identification information out of a recording medium with the cow identification information recorded therein. A somatic cell concentration measuring section measures the somatic cell concentration of milk obtained from a cow. A measurement result recording section records the somatic cell concentration and the cow identification information in association with each other. The recording medium is attached to the cow.

According to another aspect of the present invention, an identification information readout apparatus includes: an identification information readout section arranged to read cow identification information out of a recording medium with the cow identification information recorded therein; an electrical conductivity measuring section arranged to measure the electrical conductivity of milk obtained from a cow; and a measurement result recording section arranged to record the electrical conductivity and the cow identification information in association with each other, wherein the recording medium is attached to the cow.

According to the thus constructed identification information readout apparatus, an identification information readout section reads cow identification information out of a recording medium with the cow identification information recorded therein. An electrical conductivity measuring section measures the electrical conductivity of milk obtained from a cow. A measurement result recording section records the electrical conductivity and the cow identification information in association with each other. The recording medium is attached to the cow.

According to the present invention, the identification information readout apparatus may further include a measurement result output section arranged to output a recorded content within the measurement result recording section.

According to the present invention, the recording medium may be a bar code.

According to the present invention, the recording medium may be an RF tag.

According to the present invention, the recording medium may be attached to an ear of the cow.

According to the present invention, the recording medium may be attached to a leg of the cow.

According to the present invention, the identification information readout apparatus may further include: a housing having a flat surface and an intersection surface intersecting the flat surface; and a milk container provided on the flat surface, wherein the identification information readout section is provided on the intersection surface.

According to the present invention, a measuring system may include: an identification information readout apparatus according to the present invention; and an external recording apparatus arranged to record an output content from the measurement result output section.

According to the present invention, an identification information readout method includes: reading cow identification information out of a recording medium with the cow identification information recorded therein; measuring the somatic cell concentration of milk obtained from a cow; and recording the somatic cell concentration and the cow identification information in association with each other, wherein the recording medium is attached to the cow.

According to the present invention, an identification information readout method includes: reading cow identification information out of a recording medium with the cow identification information recorded therein; measuring the electrical conductivity of milk obtained from a cow; and recording the electrical conductivity and the cow identification information in association with each other, wherein the recording medium is attached to the cow.

The present invention is a program of instructions for execution by a computer to perform an identification information readout process, the process including: reading cow identification information out of a recording medium with the cow identification information recorded therein; measuring the somatic cell concentration of milk obtained from a cow; and recording the somatic cell concentration and the cow identification information in association with each other, wherein the recording medium is attached to the cow.

The present invention is a program of instructions for execution by a computer to perform an identification information readout process, the process including: reading cow identification information out of a recording medium with the cow identification information recorded therein; measuring the electrical conductivity of milk obtained from a cow; and recording the electrical conductivity and the cow identification information in association with each other, wherein the recording medium is attached to the cow.

The present invention is a non-transitory computer-readable medium including a program of instructions for execution by a computer to perform an identification information readout process, the process including: reading cow identification information out of a recording medium with the cow identification information recorded therein; measuring the somatic cell concentration of milk obtained from a cow; and recording the somatic cell concentration and the cow identification information in association with each other, wherein the recording medium is attached to the cow.

The present invention is a non-transitory computer-readable medium including a program of instructions for execution by a computer to perform an identification information readout process, the process including: reading cow identification information out of a recording medium with the cow identification information recorded therein; measuring the electrical conductivity of milk obtained from a cow; and recording the electrical conductivity and the cow identification information in association with each other, wherein the recording medium is attached to the cow.

### BRIEF DESCRIPTION OF DRAWINGS

FIG. 1 is a functional block diagram showing a configuration of an identification information readout apparatus 1 according to a first embodiment of the present invention;

FIG. 2 shows a state (FIG. 2 (a)) where a recording medium 2a is attached to an ear of a cow 2 with which the identification information readout apparatus 1 is used and a state (FIG. 2 (b)) where the recording medium 2a is attached to a leg 2c;

FIG. 3 is a front view (FIG. 3 (a)) and a plan view (FIG. 3 (b)) of the identification information readout apparatus 1;

FIG. 4 shows an example recorded content within the measurement result recording section 1d;

FIG. 5 is a flow chart showing a method of using the identification information readout apparatus 1 according to the first embodiment;

FIG. 6 is a functional block diagram showing a configuration of the identification information readout apparatus 1 according to the second embodiment of the present invention; and

FIG. 7 shows an example recorded content within the measurement result recording section 1d.

Modes for Carrying out the Invention

A description will now be given of embodiments of the present invention referring to drawings.

First Embodiment

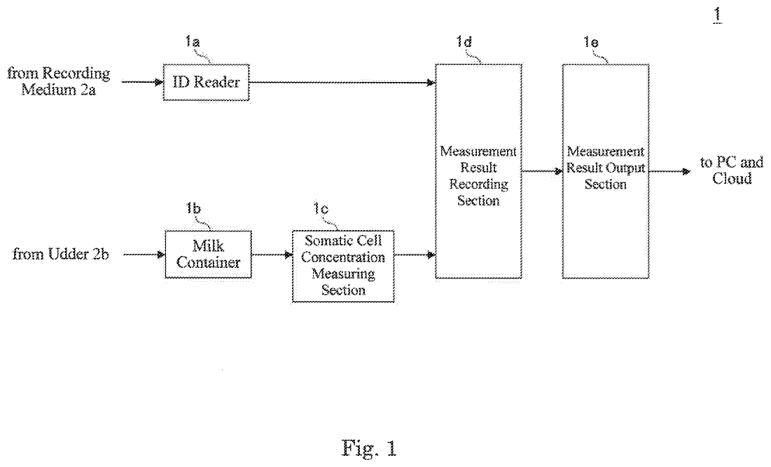

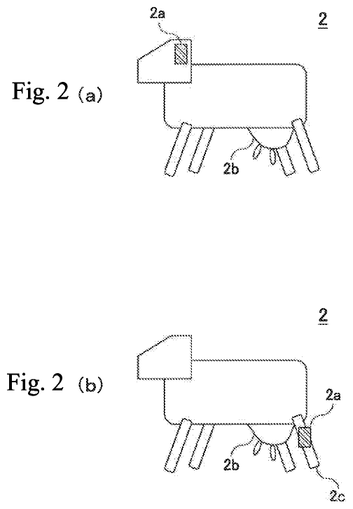

FIG. 1 is a functional block diagram showing a configuration of an identification information readout apparatus 1 according to a first embodiment of the present invention. FIG. 2 shows a state (FIG. 2 (a)) where a recording medium 2a is attached to an ear of a cow 2 with which the identification information readout apparatus 1 is used and a state (FIG. 2 (b)) where the recording medium 2a is attached to a leg 2c.

The recording medium 2a is arranged to record identification information (referred to as “ID”) for identification of the cow 2. The recording medium 2a is attached to the cow 2. The recording medium 2a may be a bar code (including a two- dimensional bar code) or an RF tag. As shown in FIG. 2 (a), the recording medium 2a may be an ear mark attached to an ear of the cow 2. Alternatively, as shown in FIG. 2 (b), the recording medium 2a may be attached to a leg 2c (e.g. hind leg) of the cow 2. It is noted that the cow 2 has an udder 2b.

The identification information readout apparatus 1 according to the first embodiment includes an ID reader (identification information readout section) 1a, a milk container 1b, a somatic cell concentration measuring section 1c, a measurement result recording section 1d, and a measurement result output section 1e.

The ID reader (identification information readout section) 1 a is arranged to read cow ID (cow identification information) out of the recording medium 2a. The ID reader 1a is, for example, a bar code reader or an RF tag reader. The milk container 1b is arranged to contain milk obtained by squeezing the udder 2b.

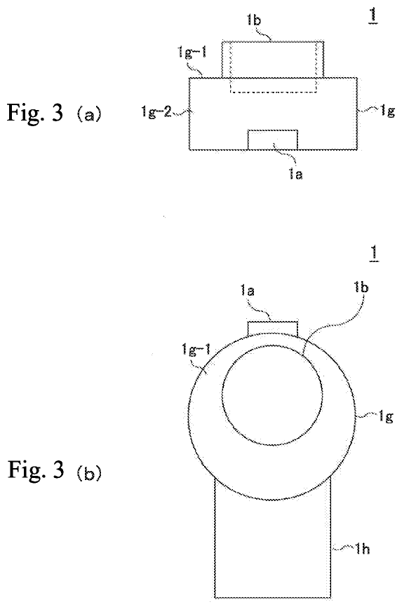

FIG. 3 is a front view (FIG. 3 (a)) and a plan view (FIG. 3 (b)) of the identification information readout apparatus 1. The identification information readout apparatus 1 has a front housing 1g and a rear housing 1h. The front housing 1g has a cylindrical shape, while the rear housing 1h has a rectangular parallelepiped shape. The front housing 1ghas a thickness greater than that of the rear housing 1h. The front housing 1g has a planar top surface (flat surface) 1g-1 and a side surface (intersection surface) 1g-2 intersecting the top surface 1g-1. The milk container 1b is provided on the top surface 1g-1. The ID reader 1a is provided on the intersection surface 1g-2. The ID reader 1a is provided in a foremost lower part of the front housing 1g.

The somatic cell concentration measuring section 1c is arranged to measure the somatic cell concentration of milk obtained from the cow 2 and contained in the milk container 1b. A somatic cell is, for example, a neutrophil cell. High neutrophil cell concentration may result from mastitis.

The neutrophil cell concentration is obtained by, for example, using a biosensor with superoxide dismutase (SOD) applied thereon and electrochemically measuring active oxygen. In addition, a reference measuring circuit not to be in contact with neutrophil cells may be provided to improve the accuracy of measurement based on comparison to a measurement value from the biosensor.

In any case, measuring the somatic cell concentration itself is well known, and the somatic cell concentration measuring section 1c will not be described in detail.

The measurement result recording section 1d is arranged to receive the somatic cell concentration from the somatic cell concentration measuring section 1c and the cow ID from the ID reader 1a. The measurement result recording section 1d is arranged to record the somatic cell concentration and the cow ID (cow identification information) in association with each other.

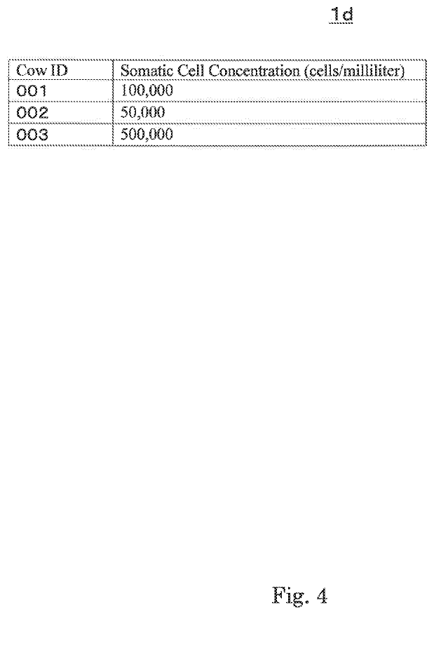

FIG. 4 shows an example recorded content within the measurement result recording section 1d. The measurement result recording section 1d records, for example, the cow ID of 001 in association with the somatic cell concentration of 100,000cells/milliliter, the cow ID of 002 in association with the somatic cell concentration of 50,000 cells/milliliter, and the cow ID of 003 in association with the somatic cell concentration of 500,000 cells/milliliter.

The measurement result output section 1e is arranged to receive and output the recorded content from the measurement result recording section 1d. The measurement result output section 1e is arranged to output the recorded content from the measurement result recording section 1d to, for example, an external recording apparatus (e.g. PC and cloud). The external recording apparatus is arranged to record the output content from the measurement result output section 1e. The capability of the measurement result output section 1e to output to the external recording apparatus may be implemented in, for example, a mobile communications terminal (e.g. smartphone). It is noted that the identification information readout apparatus 1 and the external recording apparatus constitute a measuring system.

In addition, the output from the measurement result output section 1e may be provided to and displayed on a display screen (not shown) on the rear housing 1h of the identification information readout apparatus 1.

Next will be described an operation according to the first embodiment.

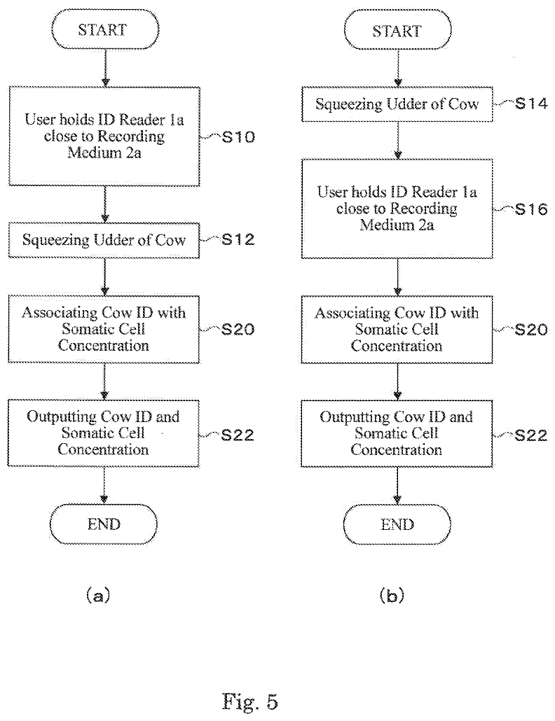

FIG. 5 is a flow chart showing a method of using the identification information readout apparatus 1 according to the first embodiment.

Referring to FIG. 5 (a), a dairyman as a user of the identification information readout apparatus 1 holds the ID reader 1a close to the recording medium 2a (S10). The ID reader 1a then reads and provides cow ID out of the recording medium 2a to the measurement result recording section 1d. Next, the dairyman squeezes the udder 2b of the cow 2 (S12). Milk obtained by squeezing is contained in the milk container 1b and its somatic cell concentration is measured with the somatic cell concentration measuring section 1c. The somatic cell concentration measured with the somatic cell concentration measuring section 1c is provided to the measurement result recording section 1d.

The measurement result recording section 1d records the somatic cell concentration and the cow ID in association with each other (S20). The measurement result output section 1e receives and outputs the recorded content from the measurement result recording section 1d to the external recording apparatus (e.g. PC and cloud) (S22).

It is noted that referring to FIG. 5 (b), the squeezing may be performed before reading ID out of the recording medium 2a. That is, a dairyman as a user of the identification information readout apparatus 1 squeezes the udder 2b of the cow 2 (S14). Milk obtained by squeezing is contained in the milk container 1b and its somatic cell concentration is measured with the somatic cell concentration measuring section 1c. The somatic cell concentration measured with the somatic cell concentration measuring section 1c is provided to the measurement result recording section 1d. Next, the dairyman holds the ID reader 1a close to the recording medium 2a (S16). The ID reader 1a then reads and provides cow ID out of the recording medium 2a to the measurement result recording section 1d. The subsequent operations (S20, S22) are the same as in FIG. 5 (a) and will not be described.

In accordance with the first embodiment, the cow ID can be read out in conjunction with the squeezing, which makes it easy to associate the somatic cell concentration measurement result with the cow. It is noted that as shown in FIG. 2 (b), the recording medium 2a, if attached to a leg 2c (e.g. hind leg) of the cow 2, comes close to the udder 2b, which makes it easier to read out the cow ID in conjunction with the squeezing than in the case where the recording medium 2a employs an ear mark (see FIG. 2 (a)).

Second Embodiment

In an identification information readout apparatus 1 according to a second embodiment, the somatic cell concentration measuring section 1c according to the first embodiment is replaced with an electrical conductivity measuring section 1f.

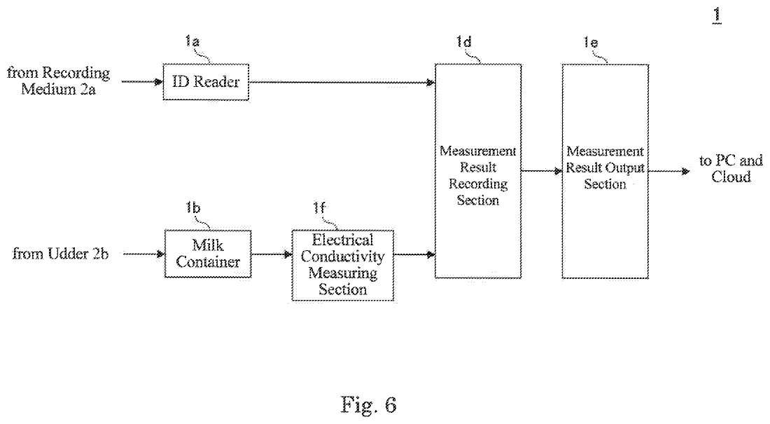

FIG. 6 is a functional block diagram showing a configuration of the identification information readout apparatus 1 according to the second embodiment of the present invention. The identification information readout apparatus 1 according to the second embodiment includes an ID reader (identification information readout section) 1a, a milk container 1b, an electrical conductivity measuring section 1f, a measurement result recording section 1d, and a measurement result output section 1e. Components identical to those in the first embodiment will hereinafter be designated by the same reference signs to omit the description thereof.

The cow 2, the recording medium 2a, the udder 2b, the leg 2c, the ID reader (identification information readout section) 1a, the milk container 1b, and the measurement result output section 1e are identical to those in the first embodiment and will not be described.

The electrical conductivity measuring section 1f is arranged to measure the electrical conductivity of milk obtained from the cow 2. Mastitis can cause the milk to have an increased amount of sodium chloride and therefore an increased electrical conductivity. Measuring the electrical conductivity itself is well known, and the electrical conductivity measuring section if will not be described in detail.

The measurement result recording section 1d is arranged to receive the electrical conductivity from the electrical conductivity measuring section 1f and the cow ID from the ID reader 1a. The measurement result recording section 1d is arranged to record the electrical conductivity and the cow ID (cow identification information) in association with each other.

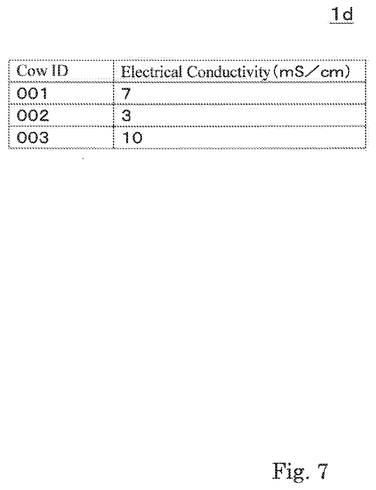

FIG. 7 shows an example recorded content within the measurement result recording section 1d. The measurement result recording section 1d records, for example, the cow ID of 001 in association with the electrical conductivity of 7 mS/cm, the cow ID of 002 in association with the electrical conductivity of 3 mS/cm, and the cow ID of 003 in association with the electrical conductivity of 10 mS/cm.

The operation according to the second embodiment is identical to that according to the first embodiment, except that the somatic cell concentration measuring section 1c is replaced with the electrical conductivity measuring section 1f and measuring the somatic cell concentration is replaced with measuring the electrical conductivity.

The second embodiment exhibits the same advantageous effects as the first embodiment.

Incidentally, the above-described embodiments may be achieved as follows. A computer including a CPU, a hard disk, and a medium (USB memory, CD-ROM, or the like) reading device is caused to read a medium with a program recorded thereon that achieves the above-described components (e.g. the measurement result recording section 1d and the measurement result output section 1e) and install the program in the hard disk. The above-described features can also be achieved in this manner.

Description of Reference Numeral

1 Identification Information Readout Apparatus

1a ID reader (Identification Information Readout Section)

1b Milk Container

1c Somatic Cell Concentration Measuring Section

1d Measurement Result Recording Section

1e Measurement Result Output Section

1f Electrical Conductivity Measuring Section

1g Front Housing

1h Rear Housing

1g-1 Planar Top Surface (Flat Surface)

1g-2 Side Surface (Intersection Surface)

2 Cow

2a Recording Medium

2b Udder

2c Leg

1. An identification information readout apparatus comprising:
an identification information readout section arranged to read cow identification information out of a recording medium with the cow identification information recorded therein;
a somatic cell concentration measuring section arranged to measure the somatic cell concentration of milk obtained from a cow; and
a measurement result recording section arranged to record the somatic cell concentration and the cow identification information in association with each other, wherein the recording medium is attached to the cow.

2. An identification information readout apparatus comprising:
an identification information readout section arranged to read cow identification information out of a recording medium with the cow identification information recorded therein;
an electrical conductivity measuring section arranged to measure the electrical conductivity of milk obtained from a cow; and
a measurement result recording section arranged to record the electrical conductivity and the cow identification information in association with each other, wherein
the recording medium is attached to the cow.

3. The identification information readout apparatus according to claim 1, further comprising a measurement result output section arranged to output a recorded content within the measurement result recording section.

4. The identification information readout apparatus according to claim 1, wherein the recording medium is a bar code.

5. The identification information readout apparatus according to claim 1, wherein the recording medium is an RF tag.

6. The identification information readout apparatus according to claim 1, wherein the recording medium is attached to an ear of the cow.

7. The identification information readout apparatus according to claim 1, wherein the recording medium is attached to a leg of the cow.

8. The identification information readout apparatus according to claim 1, further comprising:
a housing having a flat surface and an intersection surface intersecting the flat surface; and
a milk container provided on the flat surface, wherein
the identification information readout section is provided on the intersection surface.

9. A measuring system comprising:
an identification information readout apparatus according to claim 3; and
an external recording apparatus arranged to record an output content from the measurement result output section.

10. An identification information readout method comprising:
reading cow identification information out of a recording medium with the cow identification information recorded therein;
measuring the somatic cell concentration of milk obtained from a cow; and
recording the somatic cell concentration and the cow identification information in association with each other, wherein
the recording medium is attached to the cow.

11. An identification information readout method comprising:
reading cow identification information out of a recording medium with the cow identification information recorded therein;
measuring the electrical conductivity of milk obtained from a cow; and
recording the electrical conductivity and the cow identification information in association with each other, wherein the recording medium is attached to the cow.

12. (canceled)

13. (canceled)

14. A non-transitory computer-readable medium including a program of instructions for execution by a computer to perform an identification information readout process, the process comprising:
reading cow identification information out of a recording medium with the cow identification information recorded therein;
measuring the somatic cell concentration of milk obtained from a cow; and
recording the somatic cell concentration and the cow identification information in association with each other, wherein
the recording medium is attached to the cow.

15. A non-transitory computer-readable medium including a program of instructions for execution by a computer to perform an identification information readout process, the process comprising:
reading cow identification information out of a recording medium with the cow identification information recorded therein;
measuring the electrical conductivity of milk obtained from a cow; and
recording the electrical conductivity and the cow identification information in association with each other, wherein
the recording medium is attached to the cow.

16. The identification information readout apparatus according to claim 2, further comprising a measurement result output section arranged to output a recorded content within the measurement result recording section.

17. The identification information readout apparatus according to claim 2, wherein the recording medium is a bar code.

18. The identification information readout apparatus according to claim 2, wherein the recording medium is an RF tag.

19. The identification information readout apparatus according to claim 2, wherein the recording medium is attached to an ear of the cow.

20. The identification information readout apparatus according to claim 2, wherein the recording medium is attached to a leg of the cow.

21. The identification information readout apparatus according to claim 2, further comprising:
a housing having a flat surface and an intersection surface intersecting the flat surface; and
a milk container provided on the flat surface, wherein
the identification information readout section is provided on the intersection surface.

22. A measuring system comprising:
an identification information readout apparatus according to claim 16; and
an external recording apparatus arranged to record an output content from the measurement result output section.
